(19) United States
(12) Patent Application Publication
(10) Pub. No.: US 2006/0116551 A1
(43) Pub. Date: Jun. 1, 2006

(54) Single-use system and method for endoscopy pre-cleaning

(76) Inventors: Barbara Jean Lovett, Nashville, TN (US); Addam Charles Arrington, Nashville, TN (US)

Correspondence Address: Russell A. Kom;SUTHERLAND ASBILL & BRENNAN, 999 Peachtree Street, Ne, Atlanta, GA, 30309-3996, US

(21) Appl. No.: 10/999,573

(22) Filed: Nov. 30, 2004

(51) Int. Cl. A61B 1/00

(52) U.S. Cl. 600/133; 600/101

(57) ABSTRACT

A single-use cleaning system for pre-cleaning an endoscope including a container, a premixed enzymatic solution residing within the container for cleaning the endoscope, a lid removably affixed to the container to secure the premixed enzymatic solution within the container, and a towelette packet removably affixed to the lid. The towelette packet may include a disinfecting wipe for cleaning contaminates off of the endoscope tube.

### FIELD OF THE INVENTION

The invention relates generally to the field of endoscopes and more specifically to a system and method for cleaning endoscopes.

### BACKGROUND OF THE INVENTION

Video cameras and endoscopes are used in a variety of surgical procedures. An endoscope provides the operator with internal electro-visual inspection of a particular area of interest within a patient. Endoscopes are generally equipped with accessories that allow the operator to collect biopsy or tissue samples or repair damage through electrosurgery.

One common configuration of an endoscope includes a connector component and a control component. The control component generally includes the various operator controls for the instrument, for example controls for light and video components as well as biopsy forceps. The connector component includes connections for, among other things, the light source, the air and water inlets, the suction source, as well as the electrical connections for the videoscope cable.

Cleaning, disinfecting, and sterilization (hereinafter collectively referred to as “reprocessing”) of endoscopes allow for repeated uses of the endoscopes. The endoscope is completely waterproof as long as the integrity of the outer sheath and internal channels are not compromised. The Teflon® working channels allow easy passage of surgical instruments as long as they remain free of debris. These same working channels provide the function of supplying water to clean the lenses of the video components while inside the body as well as air to dry the lens after cleansing and suction to remove any bodily fluid or contaminant as necessary to allow a clear image. According to the Society of Gastroenterology and Nurses Associates, Inc (SGNA), the reprocessing protocol begins in the patient room. The immediate flushing of the biopsy/suction and air/water channels of the endoscope precludes drying of organic and inorganic debris on lumen surfaces and may remove large numbers of microorganisms. If the endoscope is left unclean for some time after use, dried blood, mucous, or other debris may cause damage to the instrument or may interfere with the ability of the user to properly process the endoscope.

Currently, individuals often fail to properly clean the endoscopes as required by the manufacturers instructions and industry procedure. The individuals tend to not properly wipe down the exterior of the endoscope and fail to flush the channels of the endoscope with the proper solution. The problem of needing to wipe the exterior and flush the channel in a part of the hospital, surgery center, or physicians office that is not designed for immediate cleaning of instruments leads to ad hoc use of towels to wipe the exterior of the endoscope and use of containers with staff mixed pre-cleaning solution with inadequate and sometimes unsafe labels applied to flush out the channels.

An effective, efficient, and containerized single-use product is needed to properly flush and begin the reprocessing procedure for the endoscopes used in the patient room of the hospital. Since SGNA standards require the use of a container with enzymatic detergent solution be used to clean the scopes, there is a need for a pre-made product that contains the pre-measured solution necessary for this important first step on the market and allows for quick and easy cleaning of endoscopes.

### SUMMARY OF THE INVENTION

A single-use cleaning system for pre-cleaning an endoscope including a container, a premixed enzymatic solution residing within the container for cleaning the endoscope, a lid removably affixed to the container to secure the premixed enzymatic solution within the container, and a towelette packet removably affixed to the lid. The towelette packet may include a disinfecting wipe for cleaning contaminates off of the endoscope tube.

In one aspect of the invention the cleaning system may be used to clean an endoscope device that includes a control system and an insertion tube. The control system and the insertion tube may include two channels.

In another aspect of the invention the container may be made of a translucent plastic and may by cylindrical. In a further aspect of the invention, the container is approximately 8 ounces in volume. The pre-mixed enzymatic solution may be a mixture of enzymatic cleaner and water. In another aspect of the invention, a label is affixed to the container to identify the pre-mixed enzymatic solution.

The single-use method system may be used in cleaning steps by hospital operators. The steps for using the cleaning system for pre-cleaning an endoscope includes providing an endoscope that includes an insertion tube and a control system, providing a container which holds a premixed enzymatic solution, providing a lid removably affixed to said container for securing the premixed enzymatic solution in said container, providing a towelette packet removably affixed to the lid wherein a disinfecting wipe is contained in the towelette packet, removing the disinfecting wipe from the towelette packet, wiping the insertion tube with the wipe to clean the contaminates from the outside of the tube, removing the lid of the container, inserting the insertion tube into the premixed enzymatic solution residing in the container, flushing the pre-mixed enzymatic solution through the insertion tube and control system, and discarding said towelette, container, and lid after cleaning of the endoscope. In one aspect of the invention, the pre-mixed enzymatic solution is flushed through a biopsy channel of the insertion tube and the control system.

### BRIEF DESCRIPTION OF THE DRAWINGS

FIG. 1 is a perspective view of a control section of an endoscope.

FIG. 2 is a perspective view of an embodiment of a single-use cleaning system.

FIG. 3 is a top down view of a single-use cleaning system.

FIG. 4 is a perspective view of the process of using a pre-moistened wipe to remove the gross contaminants from the endoscope tube.

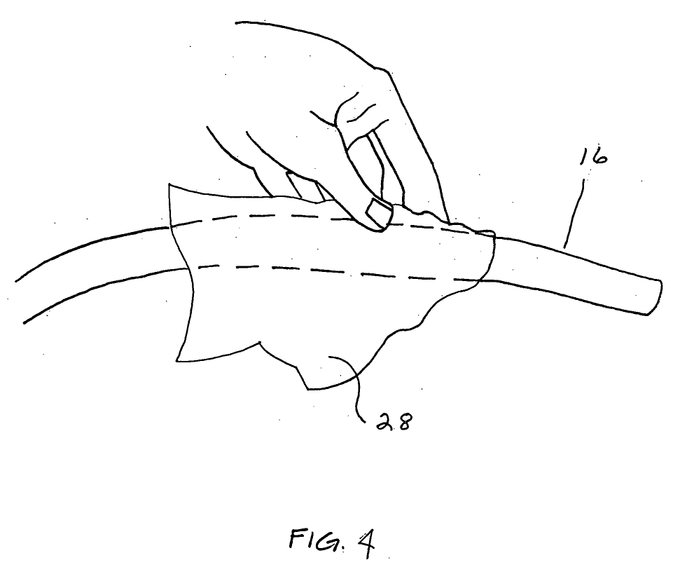

FIG. 5 is a perspective view of the pre-mixed enzymatic solution being suctioned through a working channel of an endoscope.

### DETAILED DESCRIPTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

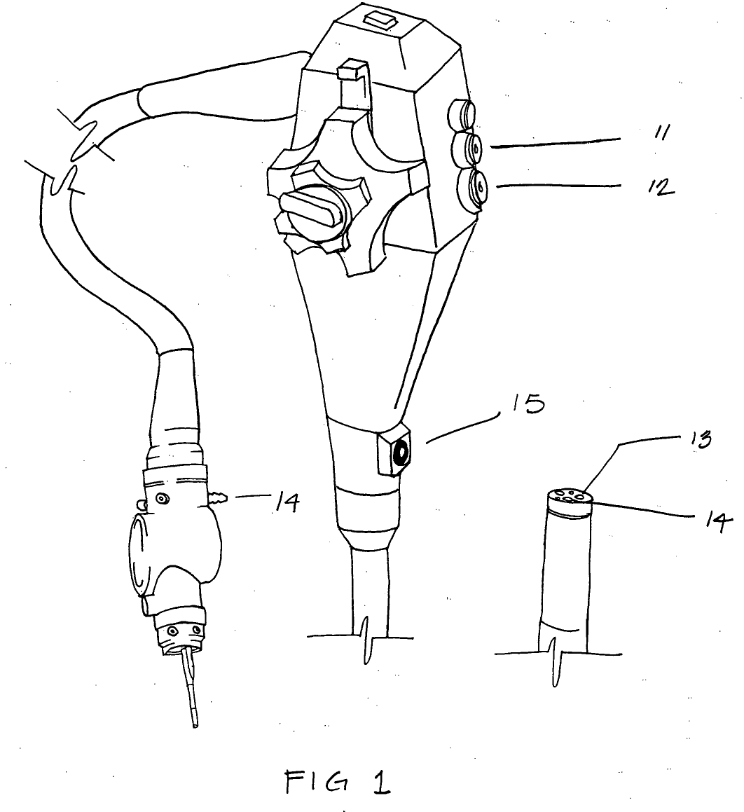

The present invention relates generally to a single-use cleaning system and method for cleaning endoscopes and other medical video and fiber optic equipment that use biopsy and working channels to perform procedures in the human body. FIG. 1 illustrates a control section 10 of a common endoscope that may be cleaned by the cleaning system. Generally, the control section 10 may contain a suction valve 11 and an air/water valve 12 that control the functions of a biopsy/suction channel 14 and the air/water channel 13. The instrument channel opening 15 accepts the biopsy forceps and other instruments into the biopsy/suction channel 14. The biopsy/suction channel 14 and air/water channel 13 may be used to suction cleaning solution into the endoscope for cleaning. The endoscope may further include an insertion tube 16 that attaches to the control system 10 of the endoscope. The insertion tube is the portion of the endoscope that is inserted into the patient. One of ordinary skill in the art will appreciate that this particular endoscope configuration is used for illustrative purposes only and this invention may be used on any endoscope with any configuration or any other medical video and fiber optic device.

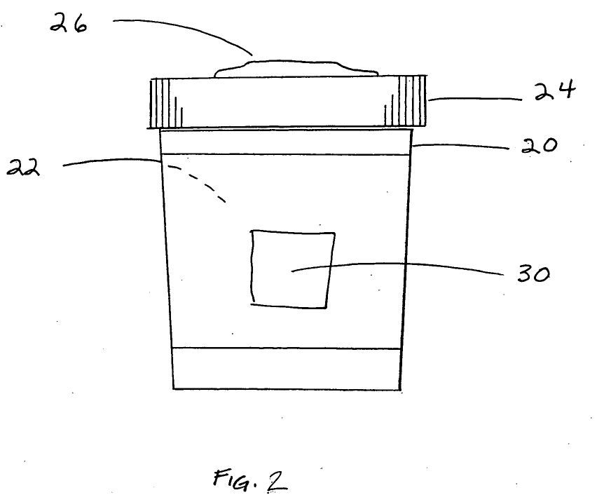

FIG. 2 illustrates a single use container 20 used in the cleaning system. The container 20 holds a pre-mixed enzymatic solution 22. The pre-mixed enzymatic solution 22 consists of any commercial medical grade concentrated enzymatic cleaning solution and filtered or unfiltered water. The pre-mixed enzymatic solution 22 is mixed at the proper ratio for cleaning endoscopes prior to being inserted into the container 20. The premixed solution prevents operators from having to mix the enzymatic cleaner with water at the proper ratio at the time of cleaning, thereby, avoiding inadvertent mixing errors and saving valuable post-procedure time in the hospital room.

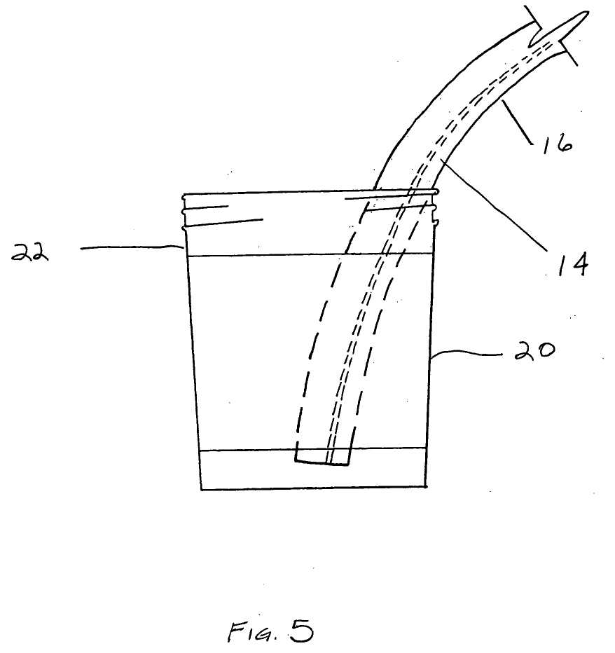

The container 20 may be substantially cylindrical to accommodate the insertion of an endoscope into the pre-mixed enzamatic solution 22. One of ordinary skill in the art will appreciate that any shape container that allows the insertion of a endoscope is contemplated such as squarely or conically shaped. The depth of the container 20 is of sufficient depth and width to adequately accept the distal end of the endoscope 10 as illustrated in FIG. 5. In an exemplary embodiment, the volume of the container in the exemplary embodiment is approximately 8 ounces. One of ordinary skill in the art would appreciate that the container may be any dimension sufficient to hold the required amount of premixed solution. In another embodiment, the container 20 is formed of translucent plastic, which assists in properly containing the fluid as well as ensuring the adequate suspension of the diluted enzymatic cleaning solution. However, any material that adequately contains the cleaning solution is contemplated herein.

The container may include a lid 24 to secure the premixed fluid in the container. The lid 24 is removably affixed to the top of the container such that it can be easily removed by an operator of the cleaning system. The lid 24 may be removably affixed using adhesive materials or may fit snugly about the container top.

A label 30 may be attached to the container 20 to allow the user to appropriately identify the container 20. The label 30 should be of adequate size for required instructions and ingredients to be listed thereon. The label 30 should also be of material that would resist ink smears and runs if it comes in contact with fluid. In an exemplary embodiment, the label states the name of the product, the instructions for use, and any warnings necessary to ensure, if followed, the safe usage in a medical setting.

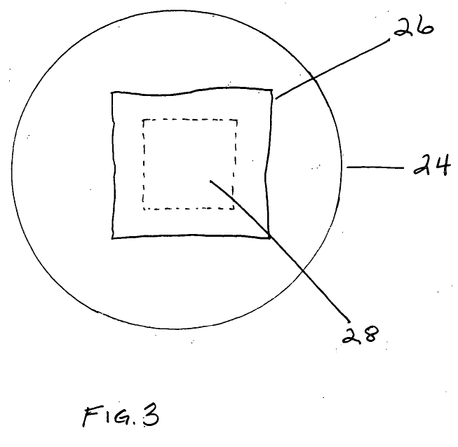

A pre-moistened disinfectant single use towelette packet 26 may be removably attached to the lid 24 of the container 20 as shown in FIGS. 2 and 3. The towelette packet 26 may be removably attached by any appropriate means such as with hot glue, Velcro, or other adhesive. The towelette packet 26 contains any commercial medical grade anti-microbial, anti-bacterial, or disinfectant wipe 28. In an exemplary embodiment, the exterior of the towelette packet 26 is properly labeled to clearly state the name of the product, instructions for use, and any warnings necessary to ensure the safe usage in a medical setting. In another embodiment, the towelette packet 26 is affixed directly to the container 20.

The cleaning system may be used by a operator, such as a nurse or hospital technician, to clean the contaminated portions of the endoscope. In use for pre-cleaning the endoscope 10, an operator opens the single use towelette packet 26 and removes the disinfecting wipe 28 contained therein. The disinfecting wipe 28 may be used in a gloved hand of the operator, in accordance with accepted health standards, to wipe the gross contaminants from the outside of the endoscope 10 insertion tube 16. After use, the disinfecting wipe 28 should be discarded in accordance with accepted health standards.

The operator then removes the lid 24 of the container 20 and inserts the flexible endoscope insertion tube 16 into the pre-mixed enzamatic cleaner 22. This pre-mixed enzamatic cleaner 22 is then suctioned and flushed through the suction/biopsy channel 14 of the endocscope for cleaning. The pre-mixed enzymatic solution is flushed through the suction/biopsy channel of the endoscope for a period of time sufficient for cleaning.

The operator then removes the endoscope insertion tube 16 from the pre-mixed enzamatic solution 22 and discards the container, pre-mixed enzamatic solution, and lid.

Although specific embodiments of the present invention have been illustrated and described in detail, it is to be expressly understood that the invention is not limited thereto. The above detailed description of the embodiment is provided for example only and should not be construed as constituting any limitation of the invention. Modifications will be obvious to those skilled in the art, and all modification that do not depart from the spirit of the invention are intended to be included within the scope of the appended claims.

I claim:

1. A single-use cleaning system for pre-cleaning an endoscope comprising:
a container;
a premixed enzymatic solution residing within the container for cleaning the endoscope;
a lid removably affixed to the container to secure the premixed enzymatic solution within the container; and
a towelette packet removably affixed to the lid.

2. The system of claim 1, wherein said endoscope comprises a control system and an insertion tube.

3. The system of claim 2, wherein said control system comprises a biopsy channel and an air/water channel.

4. The system of claim 2, wherein said insertion tube comprises a biopsy channel and an air/water channel.

5. The system of claim 1, wherein said container comprises a translucent plastic.

6. The system of claim 1, wherein said container is substantially cylindrical.

7. The system of claim 1, wherein said pre-mixed enzymatic solution comprises a mixture of enzymatic cleaner and water.

8. The system of claim 1, wherein said container is 8 ounces in volume.

9. The system of claim 1, wherein a label is affixed to the container to identify the pre-mixed enzymatic solution.

10. The system of claim 1, wherein said towelette packet comprises a disinfecting wipe.

11. The system of claim 1, wherein said towelette packet comprises instructions for use printed thereon.

12. A method for cleaning a contaminated endoscope comprising the steps of:
providing an endoscope comprising a control section and an insertion tube;
providing a container;
providing a premixed enzymatic solution in said container;
providing a lid removably affixed to said container for securing said premixed enzymatic solution in said container;
providing a towelette packet removably affixed to the lid wherein said towelette packet comprises a disinfecting wipe;
removing the disinfecting wipe from the towelette packet;
wiping the endoscope with the wipe to clean the contaminates from the outside of the insertion tube;
removing the lid of the container;
inserting the insertion tube into the premixed enzymatic solution residing in the container;
flushing the pre-mixed enzymatic solution through the insertion tube and control system; and
discarding said towelette, container, and lid after cleaning of the endoscope.

13. The method of claim 12, wherein said premixed enzymatic solution is flushed into a biopsy channel of the insertion tube.

14. The method of claim 12, wherein said premixed enzymatic solution is flushed into a biopsy channel of the control system.
